(19) United States
(12) Patent Application Publication
(10) Pub. No.: US 2011/0160615 A1
(43) Pub. Date: Jun. 30, 2011

(54) Swallowing Frequency Measurement

(75) Inventor: Masafumi Matsumura, Neyagawa (JP)

(73) Assignee: EMPIRE TECHNOLOGY DEVELOPMENT LLC, Wilmington, DE (US)

(21) Appl. No.: 12/648,754

(22) Filed: Dec. 29, 2009

(51) Int. Cl. A61B 5/103

(52) U.S. Cl. 600/587

(57) ABSTRACT

Implementations for measurements of swallowing frequency are generally disclosed.

### BACKGROUND

In a society that may have an increasing proportion of aging individuals or the handicapped, there may be a corresponding increase in the number of individuals receiving elderly care, medical services, rehabilitation, and/or handicapped care. In such a society, it may be useful to establish an enhanced care for managing the eating functions for such individuals.

For example, impaired eating functions, eating disorders, impaired swallowing functions, swallowing disorders, and/or the like may cause aspiration pneumonia, suffocation, dehydration, or malnutrition. Further, such disorders may deprive individuals of the joy of fulfilling the basic human need of eating, potentially lowering the quality of life (QOL) for such individuals.

### BRIEF DESCRIPTION OF THE DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

In the drawings:

FIG. 1 illustrates a schematic diagram of an example swallowing monitoring system that may be arranged to monitor a user;

FIG. 2 schematic diagram of an example swallowing monitoring system that may be arranged to monitor a user;

FIG. 3 illustrates a schematic diagram of another example swallowing monitoring system that may be arranged to monitor a user;

FIG. 4 illustrates a perspective diagram of a further schematic diagram of an example swallowing monitoring system that may be arranged to monitor a user;

FIG. 5 illustrates an example process for swallowing frequency measurement;

FIG. 6 illustrates an example chart of sound data versus time;

FIG. 7 illustrates an example chart of filtered sound data versus time;

FIG. 8 illustrates an example chart of binarized sound data versus time;

FIG. 9 illustrates an example chart of non-swallowing sound data versus time;

FIG. 10 illustrates an example computer program product;

FIG. 11 is a block diagram illustrating an example computing device, all arranged in accordance with the present disclosure;

FIG. 12 illustrates an example chart of length of the silent period before swallowing;

FIG. 13 illustrates an example chart of length of the silent period after swallowing;

FIG. 14 illustrates an example chart of width of the pulse; and

FIG. 15 illustrates an example chart of frequency of the pulse.

### DETAILED DESCRIPTION

The following description sets forth various examples along with specific details to provide a thorough understanding of claimed subject matter. It will be understood by those skilled in the art, however, that claimed subject matter may be practiced without some or more of the specific details disclosed herein. Further, in some circumstances, well-known methods, procedures, systems, components and/or circuits have not been described in detail in order to avoid unnecessarily obscuring claimed subject matter. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

This disclosure is drawn, inter alia, to methods, apparatus, systems and/or computer program products related to measurement of swallowing frequency.

In some cases, a swallowing disorder may start with a reduction in swallowing function, which may be partly caused by disuse atrophy of the organs related to swallowing. Such disuse atrophy may be caused by a reduction in the number of swallows, which may weaken muscles related to swallowing, such as the tongue muscle and levator veli palatini muscle.

As will be described in greater detail below, in some instances it may be useful to provide a swallowing monitoring system for easily and automatically counting swallowing occurrences of an individual, for example.

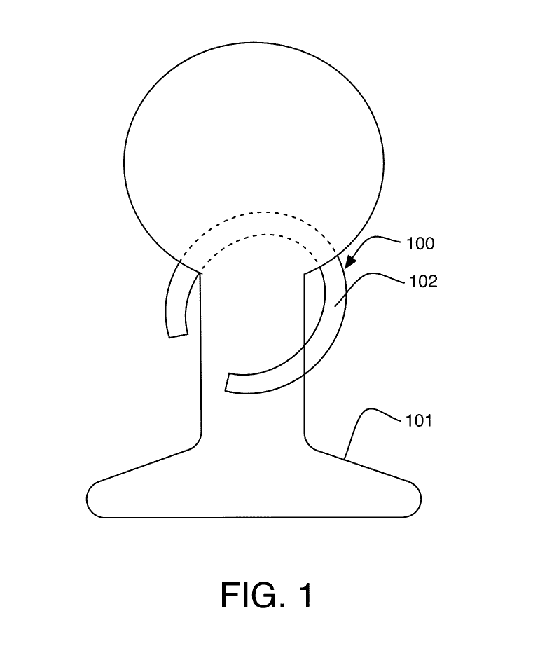

FIG. 1 illustrates an example swallowing monitoring system 100 that may be arranged to monitor a user 101, in accordance with at least some embodiments of the present disclosure. In the illustrated example, the swallowing monitoring system 100 may include a sensor unit 102 that may be configured to capture sounds of a user 101.

The swallowing monitoring system 100 may also be configured to analyze sound data received from the sensor unit 102 to execute a series of processing operations in response to the detection of sound data from the sensor unit 102. Such a swallowing monitoring system 100, may monitor sound data of a user and determine whether the sound data has characteristics of swallowing sound, based at least in part on a sound waveform of the sound data from sensor unit 102. Such operations will be described in greater detail below.

As illustrated, sensor unit 102 may be adapted to be attached to or worn by user 101. For example, sensor unit 102 may include an elastic collar configured to removably secure sensor unit 102 to a neck portion of a user 101. In some examples, such an elastic collar may be configured to have a mechanism for adjusting a collar length so as to vary in size to adjust to the neck of user 101. For example, a mechanism in which two or more collar portions (not shown) are engaged so that the two or more collar portions can adjustably mate with each other, such as a mechanism that is employed in conventional headphones or the like.

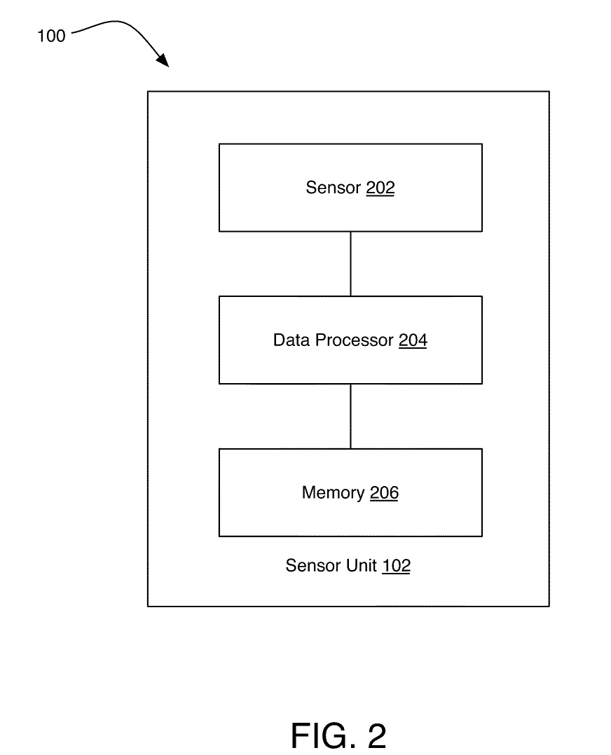

FIG. 2 illustrates a schematic diagram of an example swallowing monitoring system 100 that may be arranged to monitor a user 101, in accordance with at least some embodiments of the present disclosure. In the illustrated example, sensor unit 102 may include a sensor 202. In some examples, sensor 202 may be a contact microphone-type sensor, or the like, capable of capturing sounds of a user. In some examples, sensor 202 may be coupled to the elastic collar of sensor unit 102 and oriented to be adjacent the neck portion of a user when the elastic collar is removably secured.

Sensor unit 102 may also include a data processor 204 operably coupled to sensor 202. Data processor 204 may be configured to determine whether the sound data has characteristics of swallowing sound, based at least in part on a sound waveform of the sound data from sensor 202. Data processor 204 may be configured to distinguish the sound of swallowing from among other oral and/or throat sounds. A sensor 202 attached around the neck of a user may capture not only sounds of swallowing, but may also capture other oral and/or throat sounds, such as the sounds which come from talking, laughing, coughing, chewing, snoring, and breathing. Therefore, swallowing monitoring system 100 may be adapted to distinguish the sound of swallowing from among other oral and/or throat sounds by determining whether the sound data has characteristics of swallowing sound in accordance with one or more of programmed rules.

For example, data processor 204 may be configured to determine whether the sound data has characteristics of swallowing sound in accordance with one or more programmed rules. Such an analysis of the sound data in accordance with the programmed rules may be based at least in part on one or more threshold values and/or value ranges. Such rules, threshold values, and/or value ranges may be stored in a memory 206 operably associated with data processor 204. In some examples, such characteristics of swallowing sound may include a period of silence before a sound event, a period of silence after the sound event, pulse width of the sound event, and/or a number of pulses.

In some examples, data processor 204 may be configured to determine whether the sound data has characteristics of swallowing sound in accordance with one or more of the following programmed rules: a four hundred and fifty (450) millisecond or longer period of silence before a sound event, a one hundred and forty (140) millisecond or longer period of silence after the sound event, a one to sixty (1-60) millisecond pulse width of the sound event, and/or a number of pulses of from one to twenty (1-20) pulses. In one example, in cases where all four of these programmed rules are satisfied, swallowing monitoring system 100 may regard the sound data as having characteristics of swallowing sound. As will be discussed in greater detail below, the above listed programmed rules cover approximately 97.5% of swallowing sounds from experimental data. Accordingly, it will be appreciated that adjustments to or variations from the above listed programmed rules may occur where a greater or lesser percentage of swallowing sounds are desired to be captured.

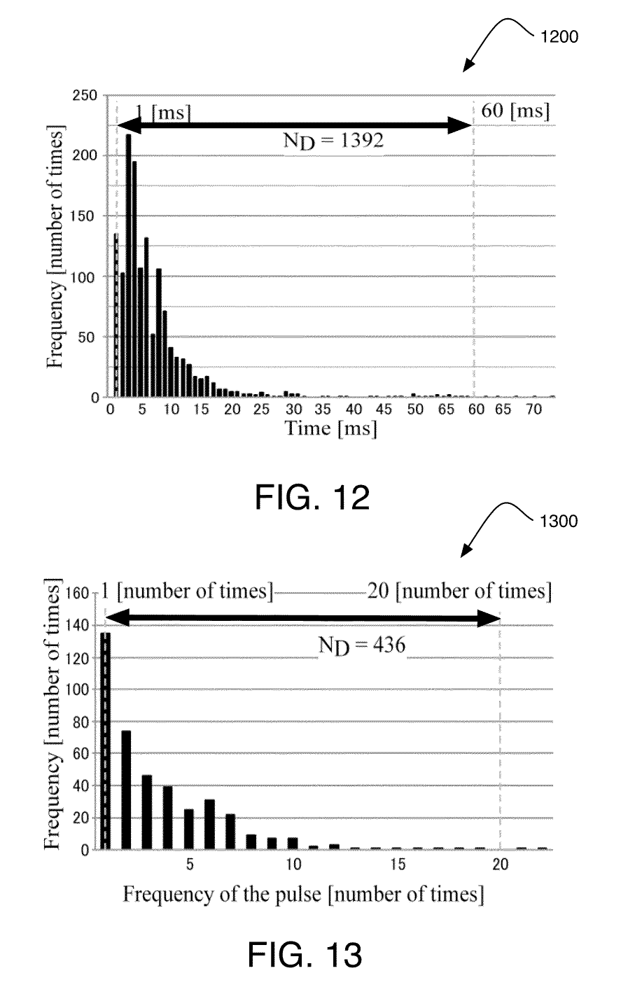

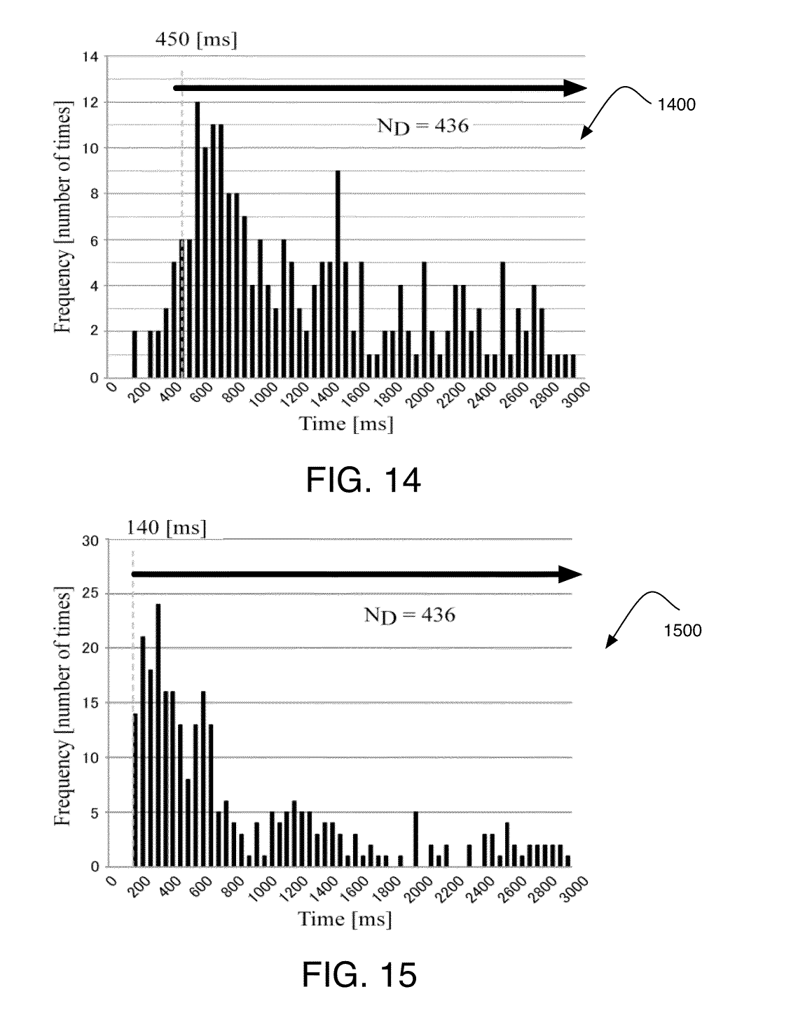

FIGS. 12-15 illustrate experimental data obtained from experimentation where 40 persons from 20s to 90s were monitored in 3496 minutes, and 436 samples of swallowing sound were obtained. Referring to FIG. 12, chart 1200 illustrates a histogram of length of the silent period before swallowing (T1). Referring to FIG. 13, chart 1300 illustrates a histogram of length of the silent period after swallowing (T2). Referring to FIG. 14, chart 1400 illustrates a histogram of width of the pulse (T3). Referring to FIG. 15, chart 1500 illustrates a histogram of frequency of the pulse (N).

Referring back to FIG. 2, data processor 204 may be configured to increment a number of swallowing occurrences based at least in part on the determination that the sound data has the characteristics of swallowing sound. Additionally or alternatively, data processor 204 may be configured to associate such counts of swallowing occurrences with a corresponding time-stamp. Such calculated data, such as counts of swallowing occurrences and/or a corresponding time-stamp, may be stored in memory 206 or in a counter. As used herein the term “counter” may refer to a device or portion of a device capable of storing the number of times a specified event has occurred. Such a counter may be a component of memory 206, a register of data processor 204, a special purpose counter-type device (not shown) operatively associated with data processor 204, or the like.

Sensor unit 102 may also include one or more additional components that are not illustrated in FIG. 2. For example, sensor unit 102 may also include a portable power source, one or more output devices, and/or one or more input devices. Such output devices may include a display or the like that may be configured to display the number of times a swallowing occurrences has occurred. Additionally, such a user interface may include one or more touch input devices, voice input devices, or the like configured to permit a user or other person to acknowledge, disable, or reset the number of times a swallowing occurrences has occurred.

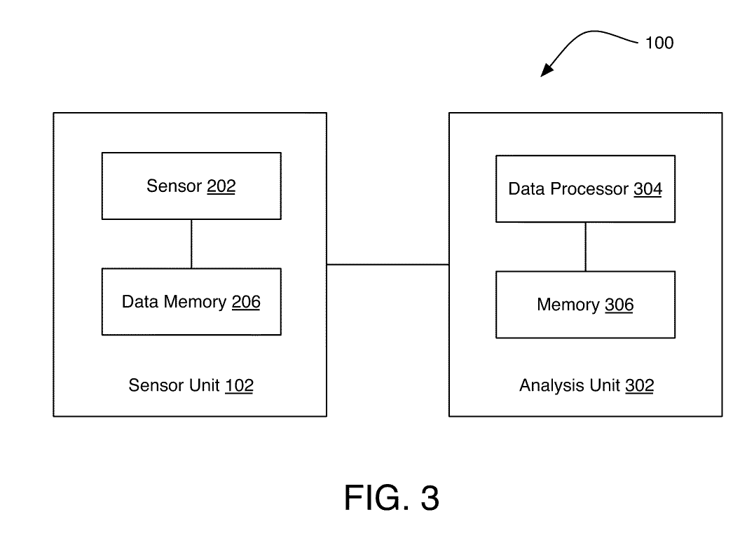

FIG. 3 illustrates a schematic diagram of another example swallowing monitoring system 100 that may be arranged to monitor a user 101, in accordance with at least some embodiments of the present disclosure. In the illustrated example, sensor 202 may be operably coupled to memory 206. Memory 206 may be configured to store such sound data for retrieval and analysis by a separate analysis unit 302.

In the illustrated example, swallowing monitoring system 100 may include analysis unit 302. Analysis unit 302 may include a data processor 304, which may be configured to determine whether the sound data has characteristics of swallowing sound, based at least in part on a sound waveform of the sound data from sensor unit 102. Data processor 304 may also be configured to increment a number of swallowing occurrences based at least in part on the determination that the sound data has the characteristics of swallowing sound. Such calculated data, may be stored in memory 306 or in a counter.

FIG. 4 illustrates a schematic diagram of a further example swallowing monitoring system 100 that may be arranged to monitor a user 101, in accordance with at least some embodiments of the present disclosure. In the illustrated example, sensor 202 may be operably coupled to a communication device 408. Communication device 408 may be configured to receive sound data from sensor 202 and send the sound data to analysis unit 302. In the illustrated example, analysis unit 302 may include a second communication device 410 configured to receive the sound data from sensor unit 102.

In one example, communication devices 408 and 410 may include short range communicators configured to transmit and/or receive data. For example, communication devices 408 and 410 may include a ZigBee-type short range communicator, Bluetooth-type short range communicator, or the like.

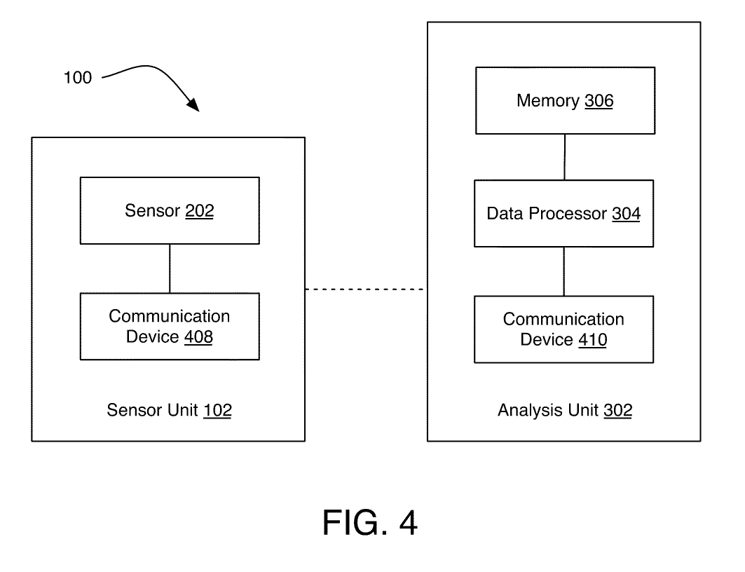

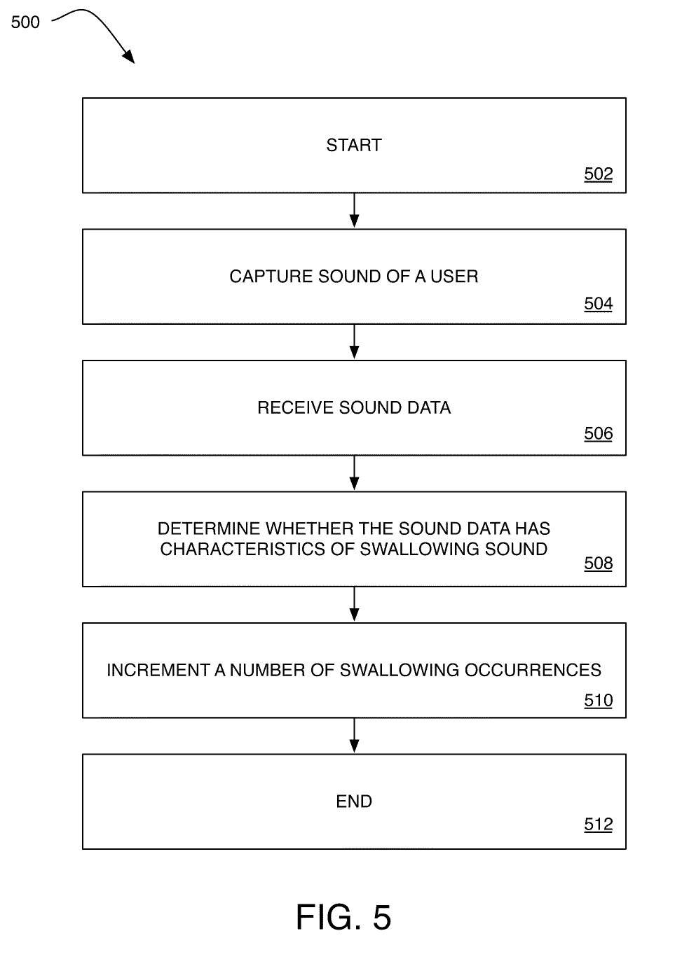

FIG. 5 illustrates an example process 500 for swallowing frequency measurement, in accordance with at least some embodiments of the present disclosure. Process 500, and other processes described herein, set forth various functional blocks or actions that may be described as processing steps, functional operations, events and/or acts, etc., which may be performed by hardware, software, and/or firmware. Those skilled in the art in light of the present disclosure will recognize that numerous alternatives to the functional blocks shown in FIG. 5 may be practiced in various implementations. For example, although process 500, as shown in FIG. 5, comprises one particular order of blocks or actions, the order in which these blocks or actions are presented does not necessarily limit claimed subject matter to any particular order. Likewise, intervening actions not shown in FIG. 5 and/or additional actions not shown in FIG. 5 may be employed and/or some of the actions shown in FIG. 5 may be eliminated, without departing from the scope of claimed subject matter. Process 500 may include one or more of operations 502, 504, 506, 508, 510, and/or 512.

As illustrated, process 500 may be implemented for swallowing frequency measurement of one or more users. Process 500 may begin at start block 502 and proceed to operation 504. At operation 504, sound of a user may be captured. For example, the sound of a user may be captured via sensor 202 (FIG. 2) (e.g. a microphone) of swallowing monitoring system 100 (FIG. 1).

At operation 506, sound data representing the sound of a user may be received from sensor 202 (FIG. 2). For example, sound data representing the sound of a user may be received from sensor 202 (FIG. 2) and stored in memory 206 (FIG. 2), memory 306 (FIG. 4), or the like.

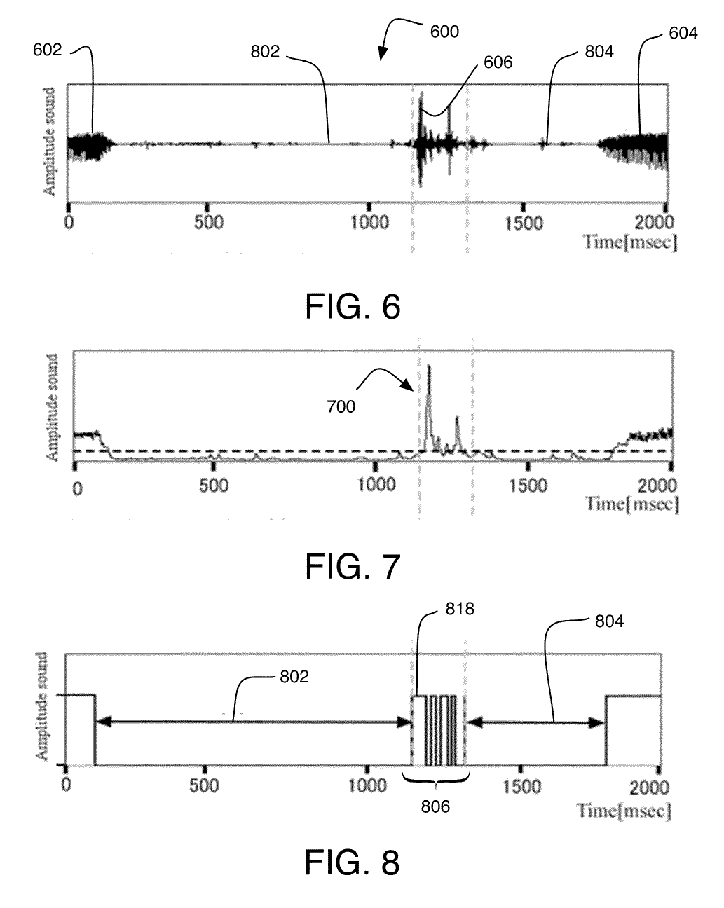

FIG. 6 illustrates an example chart of sound data versus time, in accordance with at least some embodiments of the present disclosure. As illustrated, sound data 600 representing the sound of a user may be received from sensor 202 (FIG. 2).

Referring back to FIG. 5, at operation 508, process 500 may determine whether the sound data has characteristics of swallowing sound. For example, process 500 may determine whether the sound data has characteristics of swallowing sound based at least in part on a sound waveform of the sound data.

Such a determination of whether the sound data has characteristics of swallowing sound may have several components. In one example, low-pass filtering of the sound data may be conducted. Such low-pass filtering may be capable of filtering out relatively high-frequency noise from the received sound data, and obtaining an amplitude envelope of the sound data. For example, the frequency component of swallowing sound may include a signal component of band frequency 0.8-1.5 kHz; accordingly, a 2 kHz low-pass filtering (or the like) may be capable of filtering out relatively high-frequency noise. The amplitude envelope of the sound data may be obtained by such a low-pass filtering process, as illustrated in the example of FIG. 7.

FIG. 7 illustrates an example chart of sound data versus time, in accordance with at least some embodiments of the present disclosure. As illustrated, sound data representing the sound of a user may be subject to low-pass filtering to obtain an amplitude envelope 700 of the sound data.

Referring back to FIG. 5, at operation 508, the determination of whether the sound data has characteristics of swallowing sound may include binarizing the sound data. For example, a threshold operation may be conducted on the sound data for binarization of the sound data.

FIG. 8 illustrates an example chart of sound data versus time, in accordance with at least some embodiments of the present disclosure. As illustrated, sound data representing the sound of a user may be subject to binarization 800. The illustrated binarized sound data 800 was achieved from experimental result conducted with a threshold value of 830 mV. Such a threshold value may be operative when the noise level is less than 80 dB. In this experimentation, approximately 97.5% of swallowing sounds were incorporated into the binarized sound data 800, based on the threshold value of 830 mV. Other suitable threshold values may be utilized in process 500. For example, the threshold value may be adjusted depending on the sensitivity and/or amplification degree of a given sensor 202 (FIG. 2). In the illustrated example, a threshold value represented by the horizontal dotted line in FIG. 7 may be utilized to create a pulse shape such as the example illustrated in FIG. 8.

Referring back to FIG. 5, at operation 508, the determination of whether the sound data has characteristics of swallowing sound may include calculating one or more characteristics of swallowing sound that may include a period of silence before a sound event, a period of silence after the sound event, pulse width of the sound event, and/or a number of pulses.

Referring back to FIG. 8, as illustrated, the determination of whether the sound data has characteristics of swallowing sound may include calculating one or more characteristics of swallowing sound that may include a period of silence before a sound event 802, a period of silence after the sound event 804, pulse width 806 of the sound event, and/or a number of pulses 810.

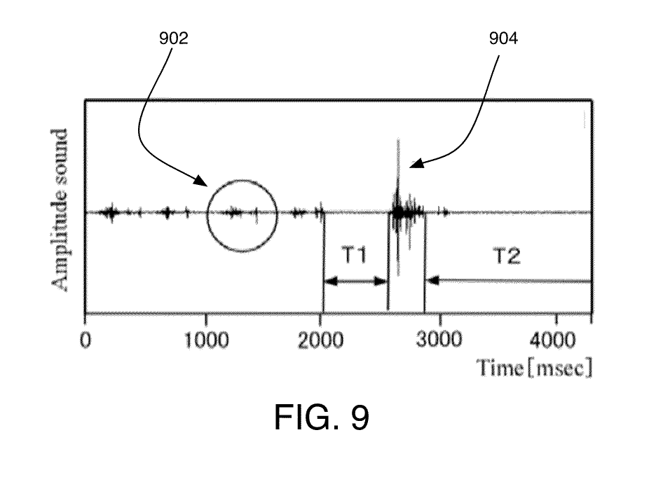

FIG. 9 illustrates an example chart of non-swallowing sound data versus time, in accordance with at least some embodiments of the present disclosure. As illustrated, sound data representing the sound of a user may include non-swallowing-type sound data 902 and/or swallowing sound-type data 904. For example sensor 202 (FIG. 2) attached around the neck of a user may capture not only sounds of swallowing, but may also capture other oral and/or throat sounds, such as the sounds which come from talking, laughing, coughing, chewing, snoring, and breathing. Therefore, process 500 may be adapted to distinguish the sound of swallowing from among other oral and/or throat sounds by determining whether the sound data has characteristics of swallowing sound in accordance with one or more of programmed rules.

Referring back to FIG. 6, an example of the waveform of sound generated when a bolus of food is swallowed is illustrated. When a bolus of food such as water is swallowed, it enters the mouth and upon reaching the throat, a swallowing sound occurs, and then it moves into the esophagus. The swallowing sound is preceded and followed by a period of silence, during which the bolus of food is moving. Items 602 and 604 shows sample mouth and throat sounds during a conversation. During a conversation, swallowing sounds 606 occur between the conversation sounds 602 and 604. Therefore speaking 602, swallowing 606, and speaking 604 sounds are shown in the waveform in this order. A period of silence 802 is observed between the conversation sound 602 and the swallowing sound 606 and the conversation sounds. Another period of silence 804 is observed between the swallowing sound 606 and the conversation sound 604.

Experimental results have shown that pulse signals with short pulse widths (several milliseconds) are observed in the interval between 200 and 300 (milliseconds) during a swallowing event 606. The characteristics of a swallowing sound can be extracted from the waveform of the sound. A series of pulse signals may be observed in a swallowing sound; accordingly, it may be possible to detect a swallowing sound based at least in part on the number of pulses that occur based on a specified the pulse time.

Referring back to FIG. 5, at operation 508, the determination of whether the sound data has characteristics of swallowing sound may include determining if the calculated characteristics of swallowing sound are in accordance with one or more programmed rules. For example, the programmed rules may include: a four hundred and fifty (450) millisecond or longer period of silence before a sound event, a one hundred and forty (140) millisecond or longer period of silence after the sound event, a one to sixty (1-60) millisecond pulse width of the sound event, and/or a number of pulses of from one to twenty (1-20) pulses. In one example, in cases where all four of these programmed rules are satisfied, process 500 may regard the sound data as having characteristics of swallowing sound.

At operation 510, process 500 may increment a number of swallowing occurrences. For example, process 500 may increment a number of swallowing occurrences based at least in part on the determination that the sound data has the characteristics of swallowing sound. Additionally or alternatively, process 500 may associate such an incremented count of swallowing occurrences with a corresponding time-stamp. Process 500 may then proceed to end block 512.

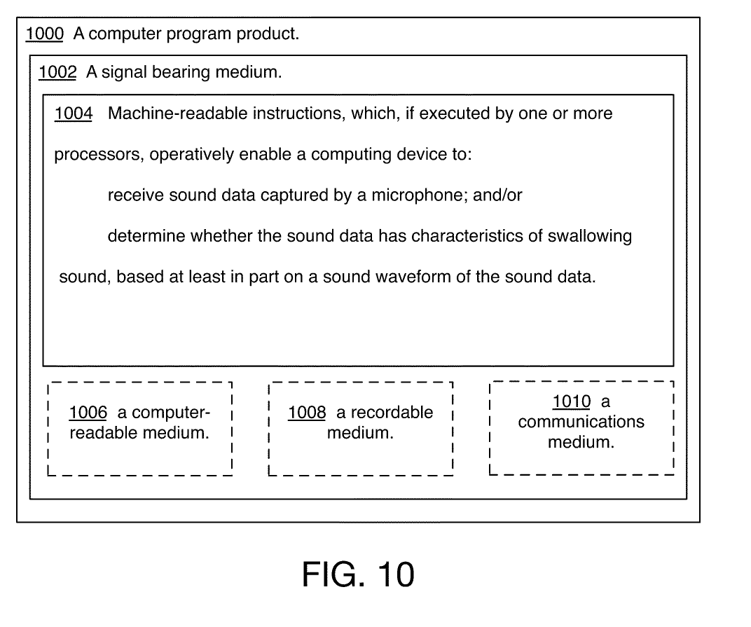

FIG. 10 illustrates an example computer program product 1000 that is arranged in accordance with the present disclosure. Program product 1000 may include a signal bearing medium 1002. Signal bearing medium 1002 may include one or more machine-readable instructions 1004, which, if executed by one or more processors, may operatively enable a computing device to provide the functionality described above with respect to FIG. 5. Thus, for example, referring to the system of FIG. 1, swallowing monitoring system 100 (FIG. 1), sensor unit 102 (FIG. 2), and/or analysis unit 302 (FIG. 3) may undertake one or more of the actions shown in FIG. 5 in response to instructions 1004 conveyed by medium 1002.

In some implementations, signal bearing medium 1002 may encompass a computer-readable medium 1006, such as, but not limited to, a hard disk drive, a Compact Disc (CD), a Digital Versatile Disk (DVD), a digital tape, memory, etc. In some implementations, signal bearing medium 1002 may encompass a recordable medium 1008, such as, but not limited to, memory, read/write (R/W) CDs, R/W DVDs, etc. In some implementations, signal bearing medium 1002 may encompass a communications medium 1010, such as, but not limited to, a digital and/or an analog communication medium (e.g., a fiber optic cable, a waveguide, a wired communications link, a wireless communication link, etc.).

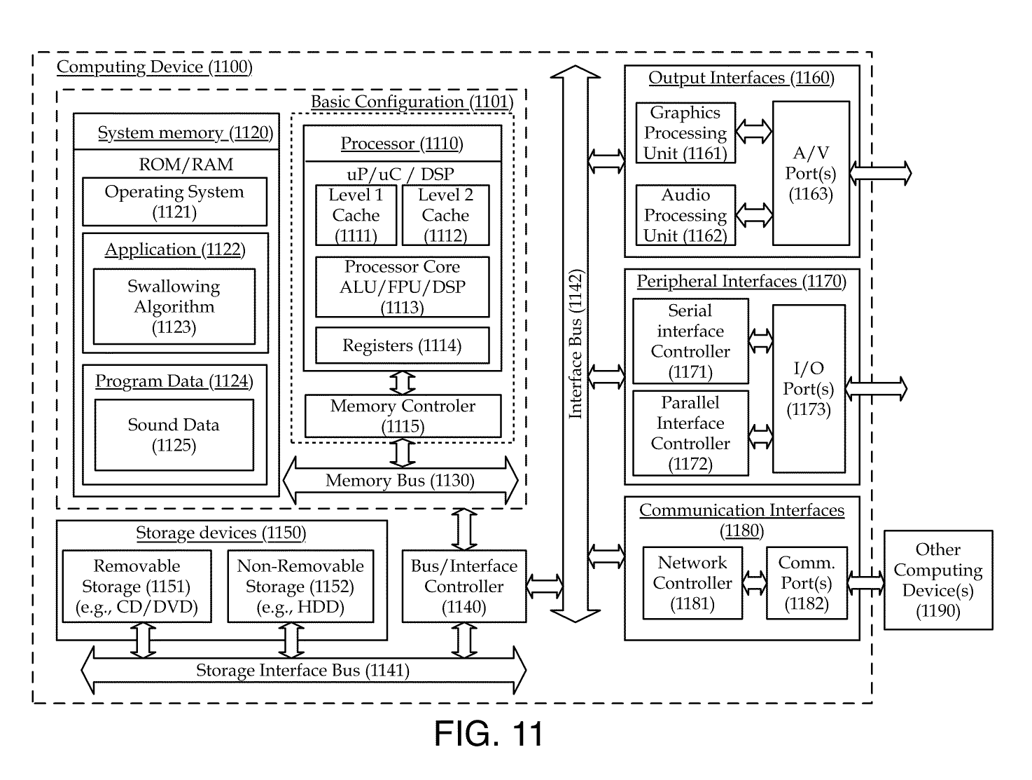

FIG. 11 is a block diagram illustrating an example computing device 1100 that is arranged in accordance with the present disclosure. In one example configuration 1101, computing device 1100 may include one or more processors 1110 and system memory 1120. A memory bus 1130 can be used for communicating between the processor 1110 and the system memory 1120.

Depending on the desired configuration, processor 1110 may be of any type including but not limited to a microprocessor (μP), a microcontroller (μC), a digital signal processor (DSP), or any combination thereof. Processor 1110 can include one or more levels of caching, such as a level one cache 1111 and a level two cache 1112, a processor core 1113, and registers 1114. The processor core 1113 can include an arithmetic logic unit (ALU), a floating point unit (FPU), a digital signal processing core (DSP Core), or any combination thereof. A memory controller 1115 can also be used with the processor 1110, or in some implementations the memory controller 1115 can be an internal part of the processor 1110.

Depending on the desired configuration, the system memory 1120 may be of any type including but not limited to volatile memory (such as RAM), non-volatile memory (such as ROM, flash memory, etc.) or any combination thereof. System memory 1120 may include an operating system 1121, one or more applications 1122, and program data 1124. Application 1122 may include a swallowing algorithm 1123 in a swallowing monitoring system 100 (FIG. 1), sensor unit 102 (FIG. 2), and/or analysis unit 302 (FIG. 3) that is arranged to perform the functions and/or operations as described herein including the functional blocks and/or operations described with respect to process 500 of FIG. 5. Program Data 1124 may include sound data 1125 for use in swallowing algorithm 1123. In some example embodiments, application 1122 may be arranged to operate with program data 1124 on an operating system 1121 such that implementations of mobile monitoring may be provided as described herein. This described basic configuration is illustrated in FIG. 11 by those components within dashed line 1101.

Computing device 1100 may have additional features or functionality, and additional interfaces to facilitate communications between the basic configuration 1101 and any required devices and interfaces. For example, a bus/interface controller 1140 may be used to facilitate communications between the basic configuration 1101 and one or more data storage devices 1150 via a storage interface bus 1141. The data storage devices 1150 may be removable storage devices 1151, non-removable storage devices 1152, or a combination thereof. Examples of removable storage and non-removable storage devices include magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include volatile and nonvolatile, removable and non-removable media implemented in any method or technology for storage of information, such as computer readable instructions, data structures, program modules, or other data.

System memory 1120, removable storage 1151 and non-removable storage 1152 are all examples of computer storage media. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 1100. Any such computer storage media may be part of device 1100.

Computing device 1100 may also include an interface bus 1142 for facilitating communication from various interface devices (e.g., output interfaces, peripheral interfaces, and communication interfaces) to the basic configuration 1101 via the bus/interface controller 1140. Example output interfaces 1160 may include a graphics processing unit 1161 and an audio processing unit 1162, which may be configured to communicate to various external devices such as a display or speakers via one or more A/V ports 1163. Example peripheral interfaces 1160 may include a serial interface controller 1171 or a parallel interface controller 1172, which may be configured to communicate with external devices such as input devices (e.g., keyboard, mouse, pen, voice input device, touch input device, etc.) or other peripheral devices (e.g., printer, scanner, etc.) via one or more I/O ports 1173. An example communication interface 1180 includes a network controller 1181, which may be arranged to facilitate communications with one or more other computing devices 1190 over a network communication via one or more communication ports 1182. A communication connection is one example of a communication media. Communication media may typically be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), infrared (IR) and other wireless media. The term computer readable media as used herein may include both storage media and communication media.

Computing device 1100 may be implemented as a portion of a small-form factor portable (or mobile) electronic device such as a cell phone, a personal data assistant (PDA), a personal media player device, a wireless web-watch device, a personal headset device, an application specific device, or a hybrid device that includes any of the above functions. Computing device 1100 may also be implemented as a personal computer including both laptop computer and non-laptop computer configurations. In addition, computing device 1100 may be implemented as part of a wireless base station or other wireless system or device.

Some portions of the foregoing detailed description are presented in terms of algorithms or symbolic representations of operations on data bits or binary digital signals stored within a computing system memory, such as a computer memory. These algorithmic descriptions or representations are examples of techniques used by those of ordinary skill in the data processing arts to convey the substance of their work to others skilled in the art. An algorithm is here, and generally, is considered to be a self-consistent sequence of operations or similar processing leading to a desired result. In this context, operations or processing involve physical manipulation of physical quantities. Typically, although not necessarily, such quantities may take the form of electrical or magnetic signals capable of being stored, transferred, combined, compared or otherwise manipulated. It has proven convenient at times, principally for reasons of common usage, to refer to such signals as bits, data, values, elements, symbols, characters, terms, numbers, numerals or the like. It should be understood, however, that all of these and similar terms are to be associated with appropriate physical quantities and are merely convenient labels. Unless specifically stated otherwise, as apparent from the following discussion, it is appreciated that throughout this specification discussions utilizing terms such as “processing,” “computing,” “calculating,” “determining” or the like refer to actions or processes of a computing device, that manipulates or transforms data represented as physical electronic or magnetic quantities within memories, registers, or other information storage devices, transmission devices, or display devices of the computing device.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In some embodiments, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a flexible disk, a hard disk drive (HDD), a Compact Disc (CD), a Digital Versatile Disk (DVD), a digital tape, a computer memory, etc.; and a transmission type medium such as a digital and/or an analog communication medium (e.g., a fiber optic cable, a waveguide, a wired communications link, a wireless communication link, etc.).

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While certain exemplary techniques have been described and shown herein using various methods and systems, it should be understood by those skilled in the art that various other modifications may be made, and equivalents may be substituted, without departing from claimed subject matter. Additionally, many modifications may be made to adapt a particular situation to the teachings of claimed subject matter without departing from the central concept described herein. Therefore, it is intended that claimed subject matter not be limited to the particular examples disclosed, but that such claimed subject matter also may include all implementations falling within the scope of the appended claims, and equivalents thereof.

What is claimed:

1. A swallowing frequency measurement method, comprising:
receiving sound data captured by a microphone; and
determining whether the sound data has characteristics of swallowing sound, based at least in part on a sound waveform of the sound data.

2. The method as recited in claim 1, further comprising:
incrementing a number of swallowing occurrences based at least in part on the determination that the sound data has the characteristics of swallowing sound.

3. The method as recited in claim 1, wherein the characteristics of swallowing sound include a period of silence before a sound event, a period of silence after the sound event, pulse width of the sound event, and number of pulses.

4. A swallowing monitoring system, comprising:
a sensor unit configured to capture sound data; and
an analysis unit configured to determine whether the sound data has characteristics of swallowing sound, based at least in part on a sound waveform of the sound data from the sensor unit.

5. A swallowing monitoring system as recited in claim 4, wherein the sensor unit further comprises a communication device configured to send the sound data to the analysis unit; and wherein the analysis unit further comprises a second communication device configured to receive the sound data from the sensor unit.

6. A swallowing monitoring system as recited in claim 4, wherein the analysis unit is configured to increment a number of swallowing occurrences based at least in part on the determination that the sound data has the characteristics of swallowing sound.

7. A swallowing monitoring system as recited in claim 4, wherein the characteristics of swallowing sound include a period of silence before a sound event, a period of silence after the sound event, pulse width of the sound event, and number of pulses.

8. A sensor unit for detecting swallowing, comprising:
an elastic collar configured to removably secure the sensor unit to a neck portion;
a sensor coupled to the elastic collar and oriented to be adjacent the neck portion when the elastic collar is removably secured, the sensor unit configured to capture sound data; and
a data processor operatively associated with the sensor unit, the data processor configured to determine whether the sound data has characteristics of swallowing sound, based at least in part on a sound waveform of the sound data from the sensor unit.

9. A sensor unit as recited in claim 8, wherein the data processor is configured to increment a number of swallowing occurrences based at least in part on the determination that the sound data has the characteristics of swallowing sound.

10. A sensor unit as recited in claim 8, wherein the characteristics of swallowing sound include a period of silence before a sound event, a period of silence after the sound event, pulse width of the sound event, and number of pulses.

11. An article comprising:
a signal bearing medium comprising machine-readable instructions stored thereon, which, if executed by one or more processors, operatively enable a computing device to:
receive sound data captured by a microphone; and
determine whether the sound data has characteristics of swallowing sound, based at least in part on a sound waveform of the sound data.
